(19) United States
(12) Patent Application Publication
(10) Pub. No.: US 2009/0048531 A1
(43) Pub. Date: Feb. 19, 2009

(54) DERMATOMAL SOMATOSENSORY EVOKED POTENTIAL (DSSEP) APPARATUS FOR REAL TIME NERVE ROOT FUNCTION DIAGNOSIS IN SURGICAL AND CLINICAL SITUATIONS

(76) Inventors: William C. McGinnis, Cincinnati, OH (US); Scott A. Metrick, Glenview, IL (US)

Correspondence Address: LADAS & PARRY LLP, 224 SOUTH MICHIGAN AVENUE, SUITE 1600, CHICAGO, IL, 60604, US

(21) Appl. No.: 12/258,587

(22) Filed: Oct. 27, 2008

Related U.S. Application Data
(62) Division of application No. 11/144,214, filed on Jun. 3, 2005.

(51) Int. Cl. A61B 5/05

(52) U.S. Cl. 600/554

(57) ABSTRACT

A dermatomol somatosensory evoked potential (DSSEP) diagnositic apparatus for evaluating nerve root functions in a mammalian subject is presented. The DSSEP apparatus includes an electrical stimulator, a stimulus site selector switchbox, a connection box, a plurality of stimulating electrodes, a plurality of recording electrodes, a computer system, and a software package. The stimulating electrodes are configured to receive and to apply an electrical stimulus onto a dermal stimulation site of the mammalian subject. The recording electrodes are configured to receive an evoked potential induced by the applied electrical stimulus such that the evoked potential response passes through nerves from the dermal stimulation site to the dermal recording site. The computer system and software package process the results.

### CROSS-REFERENCE TO RELATED APPLICATIONS

The present application is a divisional application and claims priority to U.S. patent application Ser. No. 11/144,214 filed on Jun. 3, 2005, which is incorporated herein by reference in its entirety.

### FIELD OF THE INVENTION

This invention relates to the field of neurophysiology, specifically mixed and dermatomal nerve conduction latencies and amplitudes, as well as electrophysiological evaluation of spontaneous electromyogram.

### BACKGROUND OF THE INVENTION

Somatosensory evoked potentials (SSEP) are well documented in the medical literature as neurophysiologic peripheral representations of spinal cord function. They are assessed neurophysiologically for latency and amplitude measurements that reflect mixed nerve (both sensory and motor fiber) function. These responses are averaged and a mean mathematical representation is presented as an “evoked response” or “evoked potential.” Generally, mixed nerve SSEP are robust and easily obtained from peripheral stimulation sites, and their use is well established clinically for evaluating the electrophysiological presentation in patients with neurological symptoms. Anatomically innervated by multiple overlapping nerve roots, SSEP assess mixed nerve function and cannot be used specifically to identify problems found with individual nerve roots. Thus, SSEP may be normal in patients having significant pathology in which a first DSSEP test was used to establish a baseline response of the nerve latency, followed by similar subsequent DSSEP testing to establish post-manipulation nerve latency.

Although obtaining DSSEPs is non-invasive, and relatively inexpensive, the technique is technically demanding, and reproducible results are difficult to obtain. The literature identifies the primary recording site for a dermatomal response as being over the somatosensory cortex. However, signals from the cortex are known to be ambiguous at best in both awake and in anaesthetized patients. Owen et al, (Spine vol. 18, No. 6, pgs 748-754 (1993)) in studying the differences in the levels of the DSSEP and nerve root involvement, report variable results in the peripheral innervations patterns of the dorsal nerve roots in the cervical and lumbar spine. U.S. Pat. No. 5,338,587 addressed the lack of reproducibility of responses detected at the cerebral cortex through static comparisons of transport times (latency) of signals from different stimulating electrodes.

It has been surprisingly found that superior and robust DSSEP waveforms may be recorded at a subcortical recording site. Reproducible high-confidence DSSEP data would be a considerable advance in the field.

It would also be highly advantageous to clinicians and surgeons alike to be able to compare evoked potentials in real-time and perform real-time comparisons between waveforms while they are being recorded during neurophysiological assessment, particularly intraoperatively.

### SUMMARY OF THE INVENTION

In accordance with the present invention, these and other problems are solved by the methods, computer systems, and apparatus described herein for monitoring and evaluating a neurophysiological response in a mammalian subject, specifically an evoked potential response. In one preferred embodiment of the invention, a dermatomal somatosensory evoked potential elicited from a stimulating electrode at a dermatomal site is recorded over the posterior cervical spine of a subject. In another preferred embodiment recorded evoked potentials are correlated with recorded electromyography of nerve root physiology obtained from the subject.

In a particularly preferred embodiment, recording protocols are provided for neurophysiologically assessing latency and amplitude measurements for real-time comparisons of evoked potentials being recorded, particularly comparisons to data from a normal population. Such real-time comparisons are also correlated with electromyogram data. More specifically, real-time comparisons of subcortical dermatomal somatosensory evoked potentials recorded over the posterior cervical spine of a subject, elicited from a stimulating electrode at a dermatomal site are performed. The approach may further comprise correlating with evoked potentials with electromyogram data obtained from the subject.

More specifically, the inventive approach comprises: a method of comparing and assessing evoked potentials elicited by a stimulating electrode at a stimulation site on a mammalian subject during a procedure and stored in a data storage system, the method comprising: eliciting an evoked potential response from a first stimulation site on the subject, receiving and amplifying a stimulation signal, and recording the waveform signal; automatically digitally converting the waveform signal and assigning numeric values for the absolute amplitude and absolute latency of the waveform; replicating the steps a) and b) to obtain a series of replicated digitally assigned waveform data for the given stimulation site; and mathematically conditioning the replicated digitally assigned waveform data, obtaining a validated mean value for the waveform data, then comparing the validated mean value with protocol-specific and subject-specific normal data, wherein the comparison is assessed and the deviations of the waveform data from normal noted.

Particularly, the method is applied wherein the evoked potential responses are recorded exclusively at a subcortical recording site on the subject.

An embodiment of the method is also provided for correlating the obtained waveform data with electromyogram (EMG) data obtained from the subject.

A particularly preferred embodiment is provided for comparing and evaluating the waveform data in real-time as a function of time, comprising performing a series of further trials in the above-described manner and serially comparing and evaluating in real-time the changes in the waveform data; and saving the comparisons and changes as a function of time.

It should be understood that the above inventive methods are also used with respect to not just one stimulation site but with respect to a second or a plurality of different stimulation sites.

Also provided is a computer system comprising computer-readable media having encoded instructions for executing the inventive methods as described herein.

Another preferred embodiment of the invention is apparatus for comparing and assessing evoked potentials elicited by a stimulating electrode at a stimulation site on a mammalian subject during a procedure, the apparatus comprising: hardware means for eliciting an evoked potential response from a first stimulation site on the subject, receiving and amplifying a stimulation signal, and recording the waveform signal; hardware means for automatically digitally converting the waveform signal and software means for assigning numeric values for the absolute amplitude and absolute latency of the waveform; hardware and software means for replicating the steps a) and b) to obtain a series of replicated digitally assigned waveform data for the given stimulation site; and software means for mathematically conditioning the replicated digitally assigned waveform data, obtaining a validated mean value for the waveform data, then comparing the validated mean value with protocol-specific and subject-specific normal data, wherein the comparison is assessed and the deviations of the waveform data from normal noted. An especially preferred embodiment further comprising software means for performing a series of further trials in the manner of the above described and then serially comparing and evaluating in real-time the changes in the waveform data, and for saving the comparisons and changes as a function of time.

### BRIEF DESCRIPTION OF THE DRAWINGS

FIG. 1 shows the upper extremities stimulation sites.

FIG. 2 shows the left hand stimulation sites.

FIG. 3A shows the lower extremities stimulation sites.

FIG. 3B shows the foot stimulation sites.

FIG. 4 shows the ulnar nerve stimulation site.

FIG. 5 shows the posterior cervical recording site.

FIG. 6 shows the peroneal and posterior tibial stimulation sites.

FIG. 7 shows the lumbar potential recording site.

FIGS. 8A-D show sample waveforms for the C5, C6, C7 and C8 dermatomes, respectively.

FIG. 9 shows a sample waveform for a mixed median response.

FIG. 10 illustrates schematically the methods, computer systems and apparatus of the present invention.

FIG. 11 is diagram of the patient connection box.

FIG. 12 is a diagram of the stimulus site selector switchbox.

FIGS. 13A-C illustrate electromyography recording sites for intraoperative verification of root nerves, respectively, 13A is an anterior view, 13B is an upper posterior view, 13C is a lower posterior view.

FIG. 14 depicts a flowchart of software operations.

### DETAILED DESCRIPTION OF THE PREFERRED EMBODIMENTS

### Definitions

### Somatosensory Evoked Potentials (SSEP)

Evoked potentials are the electrical summation of signals produced by the nervous system in response to electrical stimuli. Somatosensory evoked potentials (SSEP) mixed nerve responses are typically elicited by stimulation of mixed nerves at various anatomical locations, such as wrist (median), elbow (ulnar), knee (peroneal) and ankle (posterior tibial). The evoked signals are electrical impulses that are recorded from electrodes placed over the crown of the patient's head at the cerebral cortex.

### Dermatomal Somatosensory Evoked Potentials (DSSEP)

Dermatomal somatosensory evoked potentials (DSSEP) are the physiologic representation of specific nerve root function, used to evaluate sensory input from individual nerve roots. A nerve root is the proximal portion of the nerve which attaches to the spinal cord. Nerve roots are particularly prone to compression and injury by disc protrusions and other “wear and tear” changes in the spine. Nerve roots that exit the cervical and lumbosacral spine distribute in specific cutaneous skin patterns identified as dermatomes. Conventional practice utilizes recording electrodes over the somatosensory cortex on the head, with a subcortical potential recorded over the posterior cervical spine only as an adjunct site. In dermatomal somatosensory evoked potentials, specific skin sites are stimulated by a mild electrical stimulus which cases an evoked response that travels through the nerve, nerve root and spinal cord to the brain. The time (latency) taken for the evoked response to travel through the nervous system can be measured and compared to a control. If the evoked response travel time is slowed, then nerve root pathology is likely. A single dermatosensory evoked potential test procedure takes less than one minute and is repeated to assess multiple nerve roots appropriate to the particular situation.

### Electromyogram (EMG)

Measurement of an electromyogram (EMG) provides an additional neurophysiologic parameter to assess neurophysiologic function in both the clinical and intraoperative setting, founded in the fact that nerve roots distribute to both dermatomes and to muscles or myotomes. While DSSEP and the SSEP provide information about the transmission of an electrical signal from the peripheral nervous system to the central nervous system, recording and evaluating the EMG provides information about the innervation of a particular muscle (efferent) from the central nervous system to the muscle, as well as providing information about the irritability and conductivity of the muscle itself. Assessment of the nerves yields information about how the body is functioning (neurophysiologically) from the body tissue to the brain, EMG provides information about how the signal gets from the brain to the muscles that those particular nerves control. Multiple nerve roots can innervate a single muscle, and therefore obtaining DSSEP and EMG provides the clinician with a comprehensive neurophysiologic evaluation about nerve root function and the function of muscles that the nerve root innervates.

The present invention herein described comprises methods, computer systems, and apparatus for assessing nerve root function via evoked potentials, such as somatosensory evoked potentials (SSEPs) and dermatomal somatosensory evoked potentials (DSSEPs), and particularly evoked potentials recorded at a subcortical recording site. A especially preferred embodiment of the invention provides for dynamically comparing measured evoked potentials serially obtained during a procedure, and comparing them in real-time, and comparing in real-time the waveforms obtained intra-procedure with normal, control or baseline values obtained prior to the procedure.

It has been found by the inventors that DSSEPs are particularly helpful in evaluating individuals with spine and limb symptoms (i.e. pain, numbness and/or weakness). A common example is in “sciatica” of a “pinched nerve” in which a spine pain may radiate into a limb. An MRI may reveal multilevel changes and a diagnostician is uncertain which level is relevant to the patient's symptoms. DSSEPs can help make this distinction.

DSSEPs have also been found by the inventors to be helpful during surgical spinal decompression procedures. DSSEPs are performed continuously throughout the procedure and are compared in real-time to the patient's preoperative DSSEP. If an adequate decompression is accomplished by the surgeon, the previously delayed DSSEP may be seen to revert to normal or speed up which provides immediate reassurance to the surgeon. Alternatively, if the surgeon is operating near a nerve root and the DSSEP becomes delayed, then the surgeon may be alerted to the potential for injury to the nerve root by the surgery.

It will be obvious to those skilled in the art that the inventive approach may also be applied during non-surgical manipulative procedure. Therefore, it will be understood that there are many important applications of the inventive technology.

For example, real-time comparisons can be used by a surgeon to identify a particular nerve root tissue in the surgical field where it might otherwise be extremely difficult to locate and identify the anatomical position of the nerve root.

Another application of the inventive approach is as a non-invasive diagnostic device or, as a fairly rapid and non-invasive means for determining if surgery would be indicated, such applications being well-suited to provision and use in the clinical or doctor's office settings.

Further, it will be obvious that the inventive approach may provide a non-invasive means during a procedure upon a mammalian subject for development and/or testing of a medicament, or pharmaceutical and the like, or for the testing of an instrumentation or device during the design and development of medical instrument technology.

The inventors have identified a subcortical potential of lower amplitude over the posterior cervical spine, found to be a highly stable site for assessing absolute latencies and amplitudes of evoked potential waveforms when enhanced using the digital signal averaging techniques of the present invention. Therefore, one of the central aspects of the inventive approach is that the subcortical recording site, as shown in FIG. 5 (60), produces superior and robust signals whether from right side or left side stimulation, eliminating issues concerned with the drifting neurological status of the brain as well as the effects of halogen-based anesthetic agents associated with the use of cortex-derived responses.

In a highly preferred embodiment of the inventive approach, a measured evoked response from a subcortical recording site is obtained and amplified to produce a robust waveform. After analog to digital conversion of the data, quick assessment of waveforms is made because the waveforms are placed in a digital format where they can be easily measured, saved and transported, for future use and comparison. A recorded response is cursor marked for visual inspection of the wave morphology, then saved and compared with a normal response. This process is continued in sequence until the end of the testing protocol. The mathematically summated tracings (signal averaging) of the physiological responses from the recording electrodes are time-locked to a given stimulus, and replicated for a determined number of responses. A mean mathematical representation is then presented as the averaged response at those recording electrodes to the given stimulus. The evoked response is then assessed for its absolute latency and absolute amplitude. Signal amplification reduces the signal to noise ratio, improving signal averaging. As a result, substantially fewer number of replications are needed to produce robust and reliable data than is conventionally required.

All of the above described may be carried out statically or dynamically. Real-time assessments and comparisons are provided to the practitioner or surgeon for monitoring and guidance purposes, wherein waveforms are obtained serially over the course of a procedure and dynamically assessed and compared in real-time. In addition, the above-described additionally comprise static and dynamic correlation of nerve root function with electromyography of individual muscles innervated by those nerves.

In a highly preferred embodiment therefore, the present invention provides for the practitioner to compare recordings in real-time serially during a procedure being carried out upon the subject. Comparisons are made while recordings are being made with one or more normal or baseline recordings from a subject or from an asymptomatic population. The comparisons may be performed serially on sequentially obtained evoked potentials obtained throughout the course of the procedure, and assessments as well as individual waveform data may be made by any means of visual display of the previously buffered and/or stored waveform data.

It is important to realize that real-time comparisons of waveforms may be performed on both test recordings during a procedure and with respect to waveform data obtained prior to the procedure as baseline recordings from the test subject or from normal values obtained from a so-called normal population selected by the practitioner according to criteria selected by the practitioner and stored.

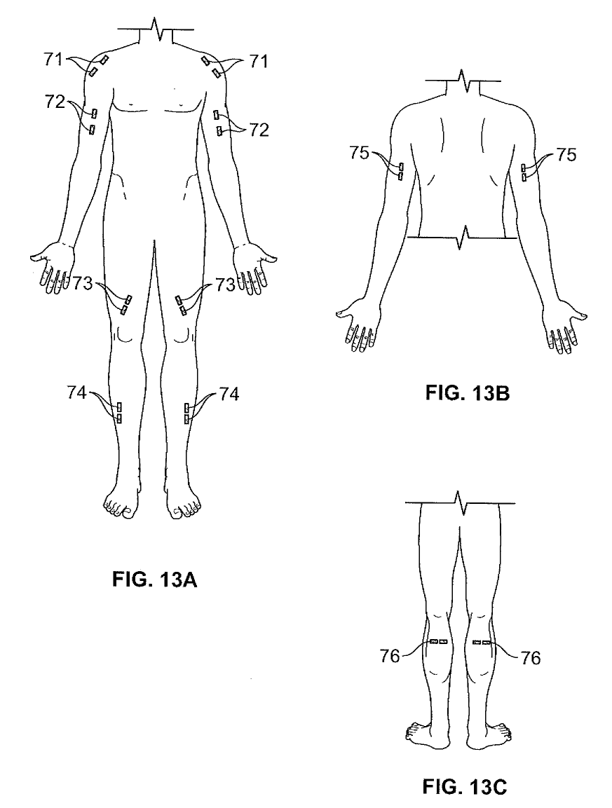

In addition to the foregoing, yet another preferred embodiment of the invention provides for correlating evoked potential data with electromyography (EMG) of nerve root physiology. Assessment of wave presentations occurring in an electromyogram may be integrated with DSSEP data for determining the function of muscles innervated by the nerve root. Such activity may be used both as a marker for stimulus and as a marker for pathology. EMG activity is assessed in free-run format, using recording sites as shown in FIGS. 13 A-C, comprising assessing a baseline waveform activity, then assessing a subsequent activity, wherein a transient increase in amplitude reflecting a muscle activity near a specific nerve root may be measured and correlated with a dermatomal evoked potential.

In another highly preferred embodiment, the inventive approach may be used to assess the adequacy and safety of a nerve root decompression during spine surgery. During surgery, the inventive approach may be used to help prevent irreversible nervous system damage. Dynamic status of the nerve roots latency during decompression as described herein provides the surgeon with a real-time assessment of the adequacy of decompression. If the latency of waveforms is delayed, when compared to normal or control values, the surgeon or practitioner may suspect a pathological process at that specific root level. Pre-decompression latency delays intraoperatively, would therefore provide the surgeon with electrophysiological evidence of nerve root compression.

A dynamic alarm can provide early warning during a surgical procedure that would warn of possible physiological insult to a neural structure and help prevent a post-operative pathological presentation. Thus, the inventive approach can be used to improve the intraoperative efficacy of a surgical intervention and help evaluate the clinical presentation of the patient. DSSEP may be performed upon a patient before surgery to provide a baseline measurement, then evoked potential responses serially obtained may be relayed and compared in real-time during the course of the surgery by the surgeon. For instance, when an impulse is obtained from a stimulation locus, particularly at a subcortical recording site, and a delayed response due to a pathological cause is recorded, and upon removal of the pathology a new recording is obtained and compared in real-time with the previous recording and/or with a pre-existing normal response time, and found to be an improved response time, the surgeon is immediately prompted as to the efficacy of the action.

A highly preferred embodiment of the invention comprises a system comprising a computer, data acquisition devices and software-driven recording and comparison protocols, for comparing and assessing the recordings in real-time. Data from responses are transported into a series of buffers for immediate recall and processing, and may be stored in temporary and permanent data storage devices. The inventive software enables the practitioner to automatically assign digital values for the absolute latencies and amplitudes of evoked potential waveforms while the recordings are taking place, automatically validate the waveform data, and dynamically compare and assess waveform data. Software also may provide and display warnings of pathological changes as they occur which may be color-coded or may be provided in any other suitable alerting form, confirms improved changes, archives data, generates reports, as well as a diverse variety of further functions described herein.

Yet another embodiment of the invention provides an icon (Haris™) which is a virtual pointer or mouse appearing on the screen at the start to prompt or guide the user through every aspect of the software, such as for example taking patient history, helping select a protocol, confirming proper electrode placement, recording a sequence of responses, data analysis, determining baselines, displaying warnings of pathological changes, data archiving and generation of reports.

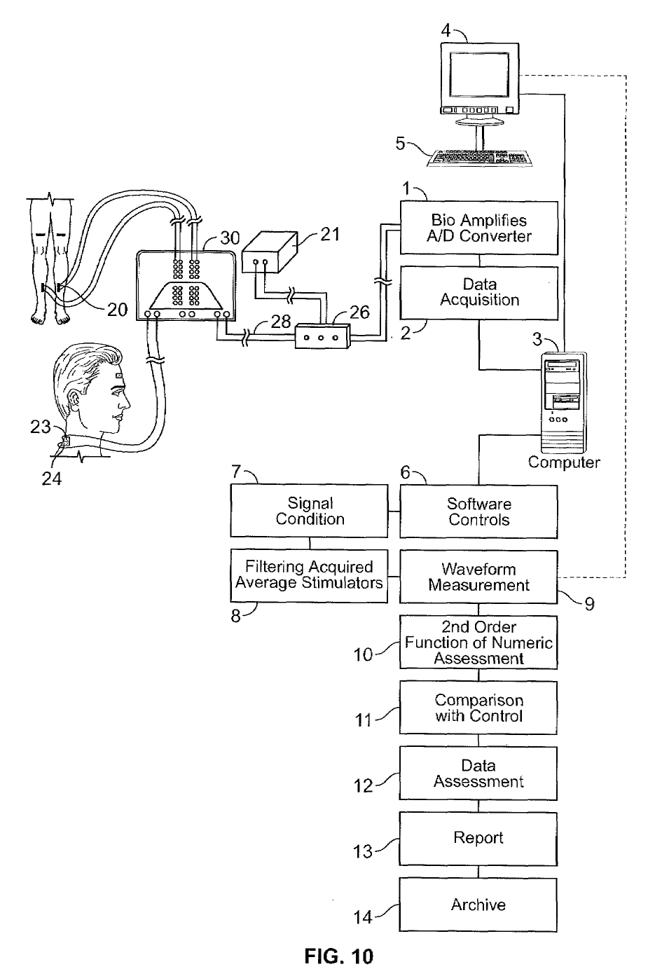

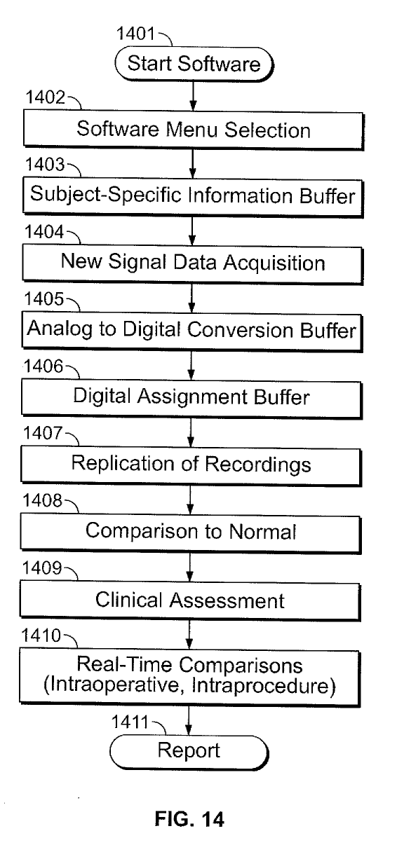

FIG. 14 depicts a flowchart of software operations for carrying out both clinical (static) settings, and intraoperative, or intraprocedural, settings in which a real-time neurological assessment of a subject being stimulated by an electrode at a dermatomal stimulation locus may be conducted as recordings are being made. It will be understood by those skilled in the art that the inventive approach assigns data being recorded to buffers for instant recall during the course of the procedure, and to permanent storage for archiving. It will also be understood by those skilled in the art that the inventive approach may have many variations on the substance of FIGS. 10 and 14 without departing from the spirit of the present inventive approach, which is to provide rapid and automatic real-time mathematical assessment of waveforms to provide dynamic and critical assessment and assurance to a practitioner during a procedure.

In FIG. 14, 1401 initiates the software. At 1401, Icon Haris™ appears onscreen to navigate the user through the inventive system. 1402 is a user menu in which protocol options are presented for selecting a recording protocol, selecting and confirming stimulation site and proper electrode placement and the like. The practitioner inputs information relating to the particular procedure being initiated, such as for example, Uppers, Lowers, Clinical, Intraoperative, etc. At 1403, subject-specific information is loaded into a buffer for later access. Here, the practitioner is prompted to input subject-specific information such as patient history and stimulation and recording parameters. The software associates the subject-specific data with data for normals from a Normal Data Buffer. The normals buffer is populated as required by the practitioner by recording the appropriate neurological data obtained from a non-symptomatic population, or by inputting the data from known subjects, including but not limited to a test subject or patient prior to a procedure. Then follows stimulation and the recording of a waveform (in analog). The stimulation signal is received and amplified. At 1404, the new signal waveform data is allocated into a protocol selection/subject history-specific allocation in a first permanent storage, and here the analog waveform may be visualized via a display. At 1405, the now protocol-specific waveform data is loaded into a Conversion Buffer for analog to digital assignment. The data is then transported into a Digital Assignment Buffer (1406). At 1406, automatic assignment of absolute amplitude is made by measuring from Marker I, representing the first linear increase, to Marker II representing the peak of linear increase, giving an absolute digital value for the amplitude of the waveform in microvolts (μV). Automatic assignment of absolute latency is made by measuring the peak of linear aggression (Marker II), giving an absolute digital value for the latency in milliseconds (ms). At 1407, replications of recordings of the waveform are performed and the data stored in serial sequence using a first order mathematical function. Variations of the normal distribution of assigned absolute digital values of greater than 1 standard deviation (sd) are reported as skewed, with a correlation coefficient of 0.90 for validation of correlation. A mathematical conditioning algorithm is used to obtain a validated mean mathematical representation of the averaged response at the given stimulating and recording electrodes. The validated mean is then assessed for its absolute latency and for its absolute amplitude. Mean validated data is then available for comparisons to normal data, normal data being mathematically conditioned and obtained in a similar manner. As will be discussed below, the number of replications required for validated mean waveform data when the present inventive approach is conducted is far fewer than conventionally required. At 1408, using a second order mathematical function, comparisons to normal are made in which the replicated protocol/subject specific data in the Digital Assignment Buffer is compared to protocol/subject specific normal data in the Normal Data Buffer. At 1409, visual display of recorded values and normal values provides the practitioner with the ability to make a clinical assessment. A report of assessments of the comparisons between recorded waveform data to normal data generated with deviations noted is automatically generated.

At 1410, using a third order mathematical function, real-time comparisons are made in which the assigned validated digital values of recorded waveforms (protocol selection/subject history-specific) residing in the Digital Assignment Buffer are compared to normals in the Normal Data Buffer. Then serial comparisons are made as function of time in the Real-time Change Buffer throughout the course of the procedure. Variations are reported as skewed deviations +/−1.0 sd. At 1411, a report is automatically generated comprising assessments of the comparisons of recorded values to normals and changes in recorded values noted as function of time. As before, visual display of the comparisons of recorded values and normal values as a function of time is provided to the practitioner throughout the course of the procedure.

It will be understood therefore by those skilled in the art that other embodiments are possible without departing from the spirit and scope of the invention and the appended claims. For example the inventive approach could comprise an apparatus for monitoring a neurophysiological response in a mammalian subject, or for determining the presence or absence of a neurological or neurophysiological condition. The inventive approach comprises a method for comparing evoked potential responses in a data storage system including a processor, memory, and multiple temporary and permanent storage devices. Alternatively, it could comprise a computer system for comparing evoked potential responses in a software-driven data storage system. In yet another embodiment, the inventive approach could comprise a computer program storage medium readable by a computing system and encoding a computer program for executing a computer process for buffering and comparing evoked potential responses in a data storage system, the computer program comprising instructions for carrying out the inventive approach as described herein. In yet another embodiment, the invention could comprise a computer data signal embodied in a carrier wave by a computing system and encoding a computer program for executing a computer process for buffering and comparing evoked potential responses in a data storage system including a processor, memory, and storage devices, the computer program comprising instructions for carrying out the inventive approach as described herein.

The basic inventive methodology utilizes three steps: i) installing electrodes on predetermined sites on the body; ii) applying an electrical charge; and iii) recording the transit time and amplitude of the charge through the body which is represented by waveforms. When the site is stimulated with an electrical stimulus, the time taken in milliseconds (ms) for the stimulus to travel to the recording electrode is recorded, multiple stimuli from the same stimulation locus are averaged, and comparisons made between validated numeric representations of the waveform.

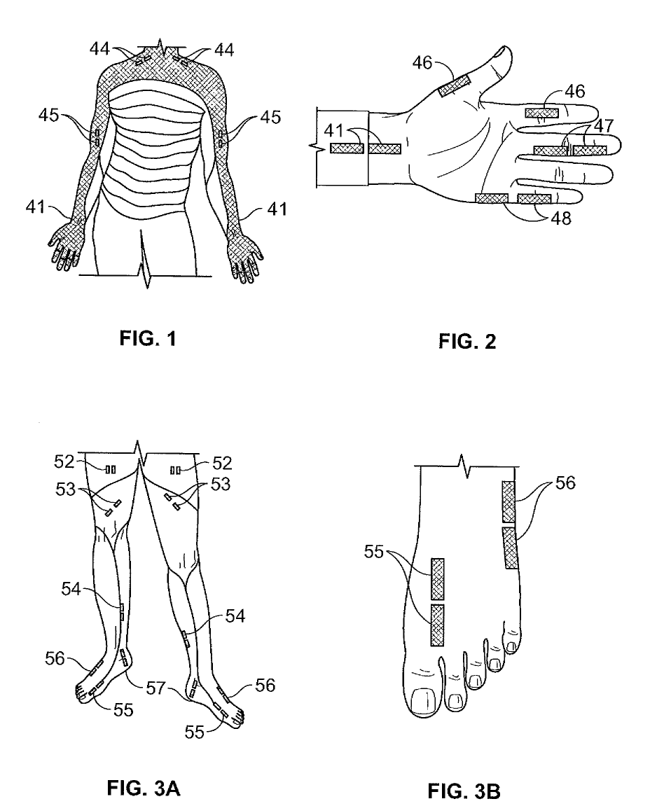

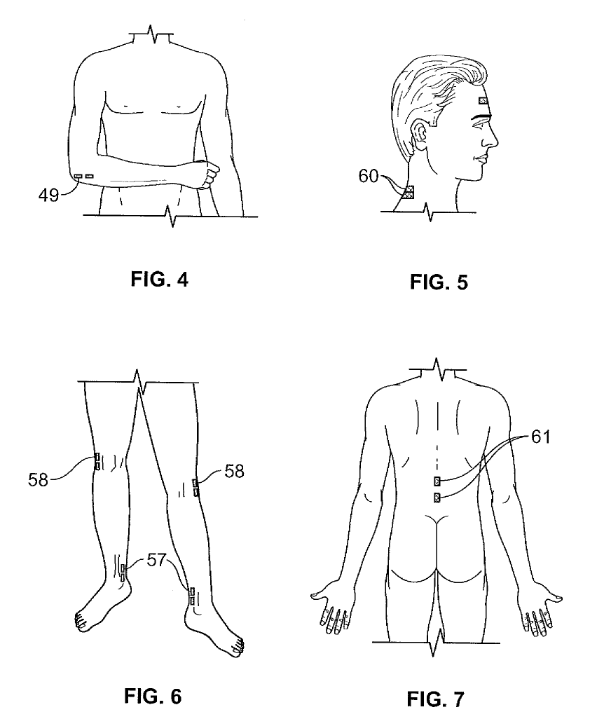

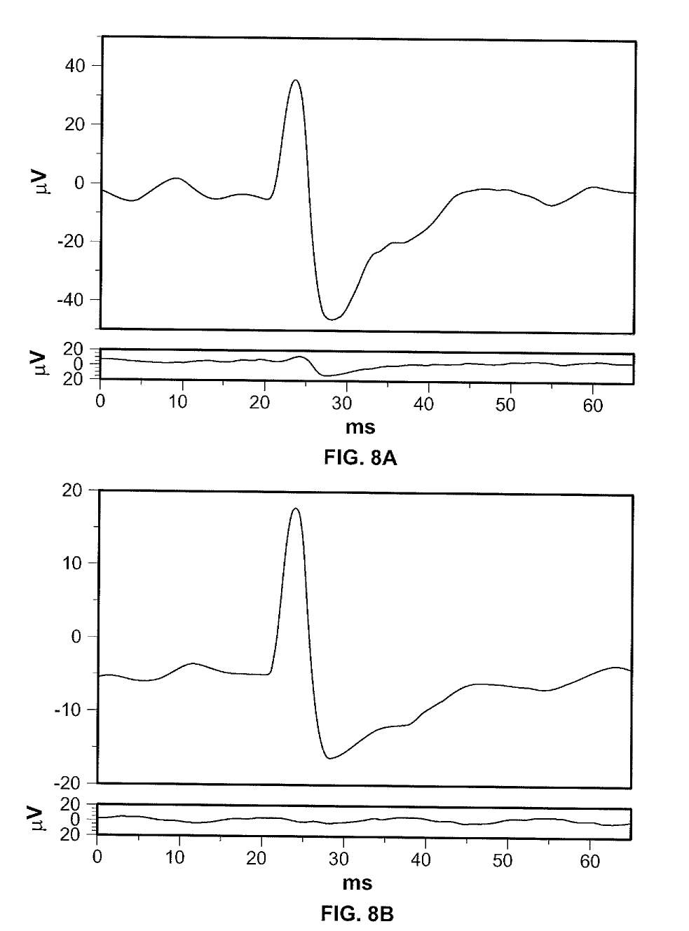

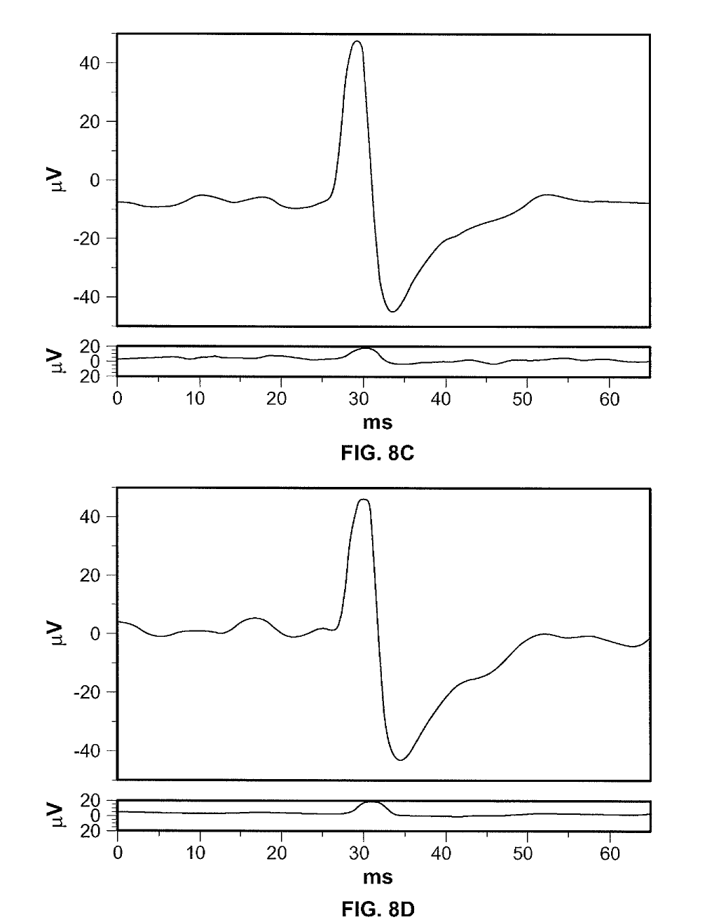

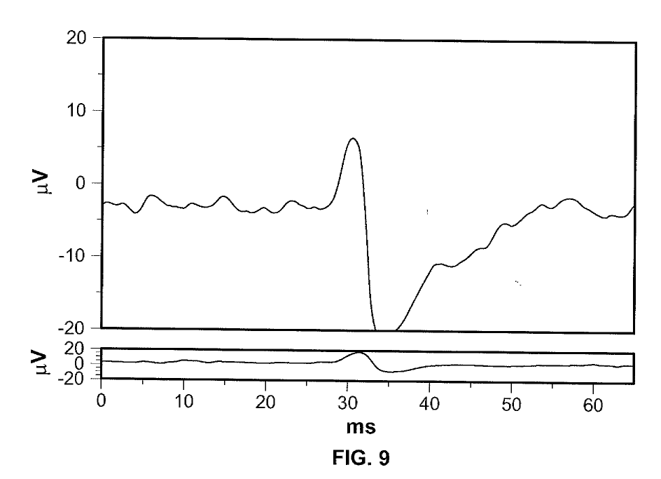

The latency of the waveforms is specifically considered using signal enhancement of distributed waveforms. Step (iii) is performed repeatedly upon a subject to elicit serial evoked responses from multiple stimulation sites which are in turn compared in real-time to the subject's normal responses and/or to control responses. Typical stimulation sites used in the present invention are shown in FIGS. 1 and 2 (upper extremities), FIGS. 3A and 3B (lower extremities), FIG. 4 (ulnar stimulation site, and FIG. 6 (posterior tibial stimulation site). FIGS. 1 and 2 illustrate the bilateral stimulation sites in the upper extremities at C4 (44), C5 (45), C6 (46), C7 (47) and C8 (48), and via the mixed median (41) (referring to reference numbers on the drawings). FIGS. 3A and 3B similarly show the lower extremities stimulation sites, L2 (52), L3 (53), L4 (54), Si (56), L5 (55) and the posterior tibial stimulation sites (57). FIG. 4 shows the ulnar nerve stimulation site (49). The position of the posterior cervical recording site is shown in FIG. 5 by reference number 60. FIG. 6 shows the positions of the peroneal stimulation sites (58) with the posterial tibial stimulation sites (57). The position of the lumbar potential recording sites is shown in FIG. 7 (61). FIGS. 8A-D and 9 show sample waveforms for C5, C6, C7, C8, and for mixed median response, respectively.

In trying to optimize the technical recording of such responses, it was discovered that significant improvement in the quality and replication of SSEP and DSSEP are achieved by the use of low stimulus intensity, greater stimulus duration, larger surface area contacts and a decreased improved amplifier signal-to-noise ratio. Stimulus artifact is reduced by employing longer stimulus durations and using thresholds well below motor response to reduce antidromic propagation.

To elicit a dermatomal response, an electrical current is applied to the skin which produces an electrical depolarization in small nerve fibers at a specific dermatomal site. Thereby, an afferent volley of depolarization passes orthodromicly through the nerve, nerve root entry and spinal cord to the somatosensory cortex. Given the small nature of the end fibers, the more fibers that can be recruited, the greater the amplitude of the mathematical representation of the individual roots innervation. It is important therefore to recruit a large number of sensory end fibers without exceeding threshold to elicit motor involvement. A robust evoked potential is achieved therefore when a greater contact area for the stimulating electrodes is used. The greater the surface area stimulated, the greater recruitment of a specific dermatomal distribution and the larger the contact area the more nerve fibers covered, increasing the opportunity for greater sensitivity. In a preferred embodiment of the invention therefore, a silver/silver chloride surface electrode with a contact area of about 2-4 inches is utilized, which is a larger surface area than conventional electrodes of about 0.9 cm ( 3/16- 12/16 inch) in diameter.

By means of the inventive software protocols the practitioner may adjust the stimulus applied to a stimulating site until optimization is achieved, enabling discernment of the exact loci for optimal stimulus. Thus, while dermatomal maps are known in the art, the inventive approach enables the prediction of exact loci within the dermatome for improved and reproducible data. Sites at which the stimulating electrodes are placed to elicit the dermatomal response may be specified by the practitioner by means of the inventive protocols.

After the electrodes are placed at the appropriate sites on the patient, the stimulating impulse is delivered to each selection site. The electrical impulse passes through the nerve, nerve root and spinal cord through the subcortical posterior cervical spine from which signal is recorded at a subcortical site (see (23, 24) in FIG. 10).

FIG. 10 exemplifies and illustrates schematically an especially preferred embodiment of the invention by which electrodes are connected to the patient, recording processes are carried out, and measured responses are assessed and compared either statically or dynamically via the real-time processes as described herein. As described above and as it will be understood by those skilled in the art, the real-time neurophysiological assessment described herein may be conducted in a clinical or surgical setting. In the surgical setting, the practitioner or surgeon is herewith provided the ability to rapidly assess the collected data, which is then compared to a patient's prior baseline recordings, or to normals obtained from a non-symptomatic population. Surgical application of continuously comparing the measured responses in a patient undergoing an operative procedure allows real-time mixed and individual nerve root function to be evaluated dynamically, throughout the course of the procedure. In addition according to the method herein described, stimulation and recording is repeated serially at each site of interest, and subsequent latency readings compared to baseline or normal latency readings. Thus, recordings indicated via electrodes positioned at for example (23, 24) in FIG. 10 can be visually observed by the attending surgeon at a display screen such as (4) on FIG. 10.

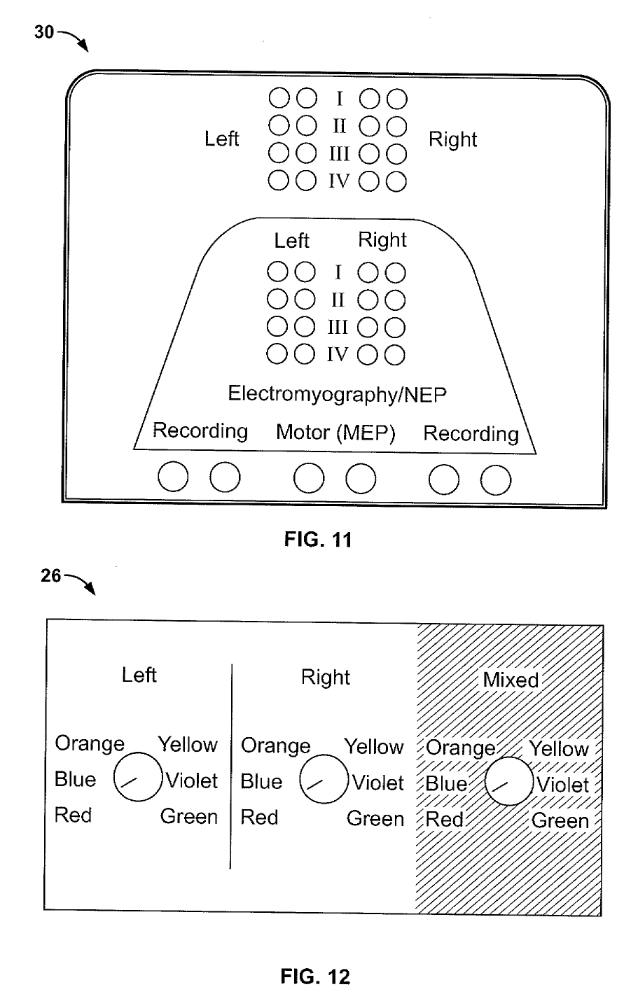

Specifically, electrodes are placed on the body in an anatomical region where the patient is symptomatic. The nerve stimulation causes evoked potentials to be generated at the electrode sites. FIG. 11 shows the patient connection box (also (30) in FIG. 10), which controls the attachment of multiple stimulating and recording electrodes to the patient. Sites on the box (shown as rings in the Figure) correlate with electrode placement on the subject: each ring in the box is a female DIN receptor receiving the male end of the appropriate electrode. The box is designated to allow both recording and stimulating sites. Recording sites for electrodes placed over the posterior cervical spine become the subcortical recording site (FIG. 5 (60)). For upper extremities, only one electrode is generally used, but for lower extremities an additional recording site to the lumbar potential (FIG. 7 (61)) is optionally available as a frame of reference to add validity to latency measurements. In a preferred embodiment of the invention, there are a total of 16 available stimulating sites, up to 8 on each side of the subject, 8 on the left, and 8 on the right, with two cortical/subcortical recording sites. For surgical evaluations and operating room settings in general, for recording both nerve root potentials and electromyography potentials, the hooded section of the box would provide for stimulating 4 left and 4 right side sites, as well as 4 recording left, and 4 recording right side EMG sites, with two (cortical/subcortical) recording sites.

FIG. 12 shows the Stimulus Site Selector Switchbox (see also FIG. 10 (26)) designed to allow control of the stimuli (two sites at a time—one left, one right) to the predetermined site on the patient connection box that provides the practitioner control over which sites are receiving the stimulus, where red is site 1, left (L1), blue is site 2, left (L2), orange is site 3, right (R3) and yellow is site 4, right (R4). In a clinical setting, the sites may be for recording or for stimulus, whereas in an operating room setting, or intraoperatively, a total of 8 stimulus channels is used. In a mixed median response for example, red is L3, blue is L4, orange is R3, yellow is R4, and violet and green are reserved for motor stimuli.

Thus as described heretofore, a pair of electrodes (20) are placed on the leg at a stimulation site selector for the L4 dermatome and the generated potentials transmitted to the patient connection box (30). The transit time from a stimulus site to the recording electrodes placed over the posterior cervical spine (23, 24) is recorded at the subcortical recording site (see FIG. 5 (60)), from which a replicable conduction latency is obtained. In principle, a single unipolar electrode can be used to obtain a recording at the subcortical recording site, but as illustrated in FIG. 10, a pair of bipolar electrodes placed at the subcortical recording site approximately 2 cm apart is an optimal configuration in the inventive approach. The posterior cervical spine recording electrodes are connected to the patient connection box (30). Stimulation site selector (26) directs the electrical impulses for stimulating the left/right, dermatomal and mixed nerve responses sites to be stimulated with electrical impulses. Conductors (28) for carrying the impulse may be copper conductors, coaxial conductors, twisted shielded conductor pairs, or the other suitable conductors. The patient connection box (30) directs impulses from the stimulation site to the specific electrode attachments to the patient. The patient connection box (30) is schematically represented in FIG. 11. Electrical stimulation (current/mA) is applied by via electrical stimulator (21) and the stimulus site selector (26), shown in FIG. 12. Stimulating electrodes (20) placed on the patient, are connected to the patient connection box (30). Output signals from bio-amplifier/A-D converter (1) to the data acquisition unit (2) via a USB connector to a computer (3) are observed on display screen (4) to which the practitioner has access via keyboard (5). Computer (3) contains data buffers to which the recordings data is transported for later assessment and comparison, and first, second, third and forth data storage devices. Boxes (6)-(14) in FIG. 10 represent operations carried out by the computer in a preferred embodiment of the invention. For example, (6) represents software controls, (7) signal conditioning, (8) software controls for low to high frequency filtering of elicited recordings, (9) waveform measurement, (10) second order transport of assigned numeric values in which the process of comparing waveform data in one database with that of another is performed, (11) in which comparison to normals or control values is carried out, (12) in which assessment of compared data takes place, (13) in which a report is generated and (14) in which reports are archived.

Baseline or normal latency control values may be obtained from a variety of different non-symptomatic population or mammalian subject sources with correction factors for height and limb length and limb temperature. The normal or control population database may be determined by each geographical location where the method is used, and may be selected according to criteria specified by the practitioner, such as for example species, race, gender, age, weight or height. Alternatively, a normal or control database may be obtained from a test subject or a patient, such as for example where measurements are made for experimental or clinical trial or research purposes, and the like. If measurements according to the inventive approach are being used to clinically or surgically evaluate an intervention, the control measurement may be made on a test subject or on a patient, prior to the intervention, where the test measurements are carried out on the same patient at or after the time of the intervention. If the measurements are being carried out to evaluate a medical instrumentation or develop such, or as part of a drug development platform, the measurements may be made on any number of control subjects and any number of different test subjects.

Following stimulation, waveforms are recorded and time-locked. To obtain a standard deviation, each impulse from a site is given a digital latency value in milliseconds (ms). The response is measured from the baseline to peak onset as the absolute latency. The peak is marked and saved as a comparative measured numeric representation, and the tracings summated or averaged.

In the inventive approach, mathematical signal enhancement is performed to produce robust waveforms in a fewer number of replications. Signal averaging is the mathematically summated tracings of the physiological responses from recording electrodes. The summated tracings are time-locked to a given stimulus (constant current mA/constant voltage/V) (duration 0.2-1.0 ms). The tracing reflects in time (ms) the detection of the evoked response, a predetermined window in milliseconds in which the response is selected, between 50 ms upper extremities and 100 ms lower extremities. The tracings are replicated for a determined number of responses. A mean mathematical representation is then presented as the averaged response at those recording electrodes to the given stimulus. The evoked response is then assessed for its absolute latency, which is the first negative occurring wave morphology, following the first positive occurring wave morphology as a function of time.

Conventionally, speaking negative polarity is up. Waveforms are further assessed for amplitude, which is measured in pV (micro volts) from the beginning of the negative wave morphology to its greatest peak. The evoked response representation is then assessed for its absolute latency, which is the first negative occurring wave morphology following the first positive occurring wave morphology as a function of time for convention (where negative is up).

The tracings are further assessed for amplitude which is measured in microvolts (μV) from the beginning of the negative wave morphology to its greater peak. The nerve stimulation causes evoked potentials to be generated at various electrode sites. These generated potentials are transmitted to patient connection box (30). By measuring the transit time from the stimulus site to the desired recording electrodes over the posterior cervical spine, FIG. 10 (23,24), a replicable conduction latency is determined, and the measured evoked response converted to a waveform tracing as described. The A-D converter (FIG. 10 (1)) allows for a quick assessment on the reading of waveforms because the waveforms are placed in a digital format where they can be easily measured, saved and transported, for future use and comparison.

A program containing a second order transport function following a pre-determined recording protocol has been written to allow the performing of real-time comparisons within the program. In one embodiment, these may be performed by the practitioner via a user-interface. A recorded response is cursor marked for visual inspection of the wave morphology. It is then saved and compared with a normal response. This process is continued in sequence to the end of the testing protocol. The test comparisons of the recorded responses are compared to normal for the evaluation of latency and amplitudes.

The range of latencies (low to high in milliseconds) for upper and lower extremities, in male and female subjects respectively, is as follows:

for upper extremities: male 31.0-39.0, female 30.0-38.0

for lower extremities: male 51.2-58.0, female 50.6-57.0

The following formula mathematically represents each bilateral upper and lower dermatomal site, where Rx=the response recorded, Ry=the known normal value for the given response, and Rx+/−Ry is <no reported change> or is,

for upper extremities:
male 3.0 ms=1.0 sd (where 10 μV=1.0 sd)
female 2.7 ms=1.0 sd (where 8 μV=1.0 sd)

for lower extremities:
male 3.2 ms=1.0 sd (where 12 μV=1.0 sd)
female 2.8 ms=1.0 sd (where 0.9 μV=1.0 sd)
   Discrepancies in latencies and amplitude have standard deviation (sd) increments from zero sd to a maximum of 3.0 sd, where normals are taken from a population database determined by each geographical location in which the method is used.

After averaging and layering, the waveforms are defined. Waveform tracings are replicated for a determined number of replications. Conventionally, some 2000 samples must normally be conducted. With the modifications according to the present invention in the recording techniques used (larger stimulation surface areas of 2-4 inches, use of the subcortical recording area, signal amplification, fewer replications with digital averaging) dermatomal nerve root response of a high technical quality is recorded with acceptable replication with between 100 and 200 recording trials conducted. According to the inventive method, a conduction latency in milliseconds (ms), is recorded from a stable site that functionally amplifies a recording site of the absolute latency +/−10 ms from the initial marked absolute latency. The signal averaging techniques are thereby enhanced to expedite the recording process, from the measured first absolute latency, a range of −10 ms to +10 ms (where the window for upper extremities is between zero and 50 ms using regular equipment) is established as a range of subsequent acceptable recorded responses to be the summated mean latency. With this step, replicate recordings of dermatomal responses can be made rapidly and of high technical quality. In the inventive approach, the standards of normality are more rigorously obtained.

For example, for the C5 cervical site, where C5x=the recorded value for that site, C5y=a corrected normal value for that site with a known +/−variance, C5y+/−C5x=C5z, and C5z=difference+/−absolute latency (with correction of the known recorded value of absolute latency +/− the known variance of that latency). If C5z is a numerical representation of a + as a latency delay, if the delay first exceeded the known positive variance in the recorded normal when compared, then it is reported as a representation of standard deviations from normal. The normal value recorded for a C5 subcortical latency is 14.7 ms+/−2.5 ms on the left side and 15.4 ms+/−1.0 ms on the right. If a C5z represents a latency delay, it is greater than 14.9+0.9=16.6 and 14.4+1.0=15.4 ms, respectively. With approximately 128 collected signal enhancement comparisons for a range of standard deviations, for a given population, C5 as 3.1 ms represents a single standard deviation. If the correct dermatomal stimulating electrodes are connected to the correct site, the site will be stimulated and compared to the normal, and the findings reported as within either a normal range or abnormal range in terms of the number of standard deviations from the normal.

In a preferred embodiment, the inventive device is configured to include a latency fail-safe feature as referred to above that alerts the practitioner if within a given set of recording data. For example, in C5, C6, C&, C8, left and right side, there might appear to be non-linear representations. For example, C5 responses should occur at approximately 14-16 ms, C6 at approximately 20-23 ms, C7 at approximately 21-23 ms, and C8 at approximately 22-24 ms. Thus, if the site at C5 has bilateral representations of between 22 and 24 ms, the software system first prompts for confirmation of electrode placement before assuming bilateral delays.

In an especially preferred embodiment of the inventive approach, the signal-to-noise ratio (s/n) is increased by means of a biosignal amplifier for EEGs (e.g. Dual Bio-Amp™, AD Instruments Pty Ltd), for electromyogram, EMG (e.g. g.tec™ Guger Technologies OEG) in conjunction with data acquisition hardware such as Power Lab™ AD Instruments Pty Ltd as the recording device. The bio-amplifier (Dual Bio Amp™) used in the inventive approach reduces the signal to noise ratio, improving signal averaging.

In theory, noise of an individual response is random with respect to the stimulus, thus the net sum of noise following the stimulus increases as n increases, where n=number of trials of time-locked recordings. The evoked response follows the same time course after each stimulus, thus there is no cancellation of this signal as responses are summated. Instead, the amplitude of the evoked response increases in direct proportion to the number of stimuli (n), and by increasing n, one is able to enhance the signal to noise ratio by the factor: n√n. By improving the signal to noise ratio, the total number of recording trials needed is reduced without skewing amplitude determinations.

In another especially preferred embodiment, FIGS. 13A-C illustrate recording sites via which recordings of spontaneous free-run EMG data by means of which verification of root nerves may be made, providing another level of physical correlation to identify the muscle that nerves innervate. In one embodiment, the EMG activity is first assessed as a baseline waveform, then subsequently as a subsequent waveform activity. The transient increase in amplitude reflecting a muscle activity near a specific nerve root is measured may be correlated with a dermatomal evoked potential. Muscle physiology reflecting the nerve root function is evaluated by inserting a needle into an appropriate muscle and observing both visual and auditory electrical muscle potentials. The amplifier is turned on and spontaneous activity may then be viewed and heard, or may be received in any other suitable electronic form. Recording electrodes are placed in the muscle via needle electrodes or over the muscle via surface electrodes, in the place where the muscle is to be evaluated. As a stored ratio of amplitude latency for a known duration, these stored samples are converted into a mathematical representation. The baseline free run EMG activity is then entered for a known muscle i.e. deltoids, biceps, triceps, bilaterally. Transient increases in amplitude for short durations in specific muscle on a specific side identifies physical activity near a specific nerve root which can be correlated with dermatomal evoked potential studies during surgery. FIG. 13A shows the positions of the deltoid (71), the bicep (72), and the quadriceps (73) recording sites. FIG. 13B shows the positions of the triceps recording site (75), and FIG. 13C, the position of the gastroci nemius recording site (76). Near nerve activity in the muscle that would show a transient linear increase, compared to a baseline recording, is then correlated with the nerve root responses, for example: deltoid C4, C5 roots in that side, bicep C5, C6, and triceps C6, C7. The root evaluation for the noted muscle would determine if there is a change of the root latency. If a change in the DSSEP waveform latency is noted, this could be correlated with changes in muscle potentials caused by root irritation, thereby providing a real-time “cross-check correlation”. One root would be identified providing irritation in the EMG, which could then further identify a specific level.

In a preferred embodiment, the inventive approach shown in FIG. 10 is followed but including use of the recording sites shown in FIGS. 13 A-C. A motor complex waveform may be identified from the background EMG activity and then used as a marker for the stimulus. The software system records several hundred milliseconds of signal following its occurrence. It then averages the intervals of occurrence to determine a baseline recording. Pathological occurrences affecting the EMG manifest as transient shorter interval train of random amplitude inter-peaks, latency are recognized as a near nerve root signal. This recognition identifies a change to the baseline intervals and a report may be generated showing the physical activity being close to a nerve, or nerve root. The trained responses of varying amplitude and random inter-peaks when compared to baseline intervals identify a mechanical stimulation, that is, pressure or traction on a nerve root. A burst response of considerable amplitude increase and a wide inter-peak latency identifies a direct contact with an innervated structure. The frequent and/or persistent occurrence of burst activity may result in neurological defects in the innervated musculature. A preferred embodiment of the invention is configured to respond to these actions by provoking a warning alert, which is prompted as an audible or color change to the recorded EMG tracing. A first reading which may act as a control reading or a baseline reading is made at the desired EMG site and the waveforms are saved for comparison when subsequent testing is performed, and the subsequent testing performed during surgery in real-time.

Thus it will be understood therefore by those skilled in the art that several embodiments of the instant invention are possible without departing from the spirit and scope of the invention and the appended claims. Such as for example, the inventive approach may be used as a diagnostic procedure, and/or in surgical management to verify surgical procedures or ascertain conditions of the body comprising for example pathologies of various locations of the body such as back, cervical spine, anterior spine, head, shoulders, pelvis, hip, leg, knee, etc. and surgeries such as for example spine surgery, hip surgery, vascular surgery (carotid, aorta etc.), tumor removal, etc. The inventive approach can be used in the doctor's office or in any clinical setting to aid evaluation of complaints such as for example back, hip, and leg problems involving compression of nerves and nerve roots, including but not limited to chronic or acute, pain, numbness, tingling, pressure, weakness, discomfort, located for example in the neck, back, hip, buttock, groin, shoulder, arm, hand, finger, leg, shin, calf, foot, toe, due to illness, trauma, accident. The approach may also be used to correlate with clinical data from x-ray, MRI, CT scan, electromyogram, steroid injection, or a drug or other therapy or intervention.

It should be understood that the present invention as described in the foregoing, may incorporate various changes, substitutions and alterations without departing from the spirit and scope of the invention as defined by the appended claims.

We claim:

1. A dermatomal somatosensory evoked potential (DSSEP) diagnostic apparatus for evaluating nerve root functions in a mammalian subject, the apparatus comprising:
an electrical stimulator for generating an electrical stimulus;
a stimulus site selector switchbox coupled to the electrical stimulator;
a connection box coupled to the stimulus site selector switchbox;
a plurality of stimulating electrodes coupled to the connection box wherein the stimulating electrodes are configured to receive the electrical stimulus and configured to apply the electrical stimulus onto a dermal stimulation site of the mammalian subject;
a plurality of recording electrodes coupled to the connection box wherein the recording electrodes are configured to receive an evoked potential response at a dermal recording site of the mammalian subject such that the evoked potential response being induced by the applied electrical stimulus at the dermal stimulation site such that the evoked potential response passes through nerves from the dermal stimulation site to the dermal recording site;
a computer system coupled to the switchbox, to the connection box, to the stimulating electrodes and to the recording electrodes, the computer system comprising:
a digitizer for digitally converting the evoked potential response into a digital waveform data;
a memory for storing the digital waveform data; and
a display terminal configured to display the digital waveform data; and
a software package driving the computer system to process the digital waveform data.

2. The DSSEP diagnostic apparatus of claim 1 further comprising an amplifier for amplifying the received evoked potential response.

3. The DSSEP diagnostic apparatus of claim 1 wherein the electrical stimulator is a component of the computer system.

4. The DSSEP diagnostic apparatus of claim 1 wherein the switchbox is manually controlled.

5. The DSSEP diagnostic apparatus of claim 3 wherein the switchbox is a component of the computer system such that the switchbox is controlled by the computer system driven by the software package.

6. The DSSEP diagnostic apparatus of claim 1 wherein the software package is a component of the computer system.

7. The DSSEP diagnostic apparatus of claim 1 wherein the software package is configured to drive the computer system to assign numeric values for an absolute amplitude and an absolute latency of the digital waveform data.

8. The DSSEP diagnostic apparatus of claim 1 wherein the software package is configured to drive the computer
to repeatedly direct the electrical stimulator to generate electrical stimuli and to apply the electrical stimuli at the stimulating electrode to elicit corresponding evoked potential responses from the dermal stimulating site to the dermal recording site;
to repeatedly detect the evoked potential responses at the dermal recording site; and
to repeatedly record the detected evoked potential responses as digital waveform data.

9. The DSSEP diagnostic apparatus of claim 1 wherein the software package is configured to drive the computer system to mathematically condition a series of digital waveform data to obtain a validated mean value from the series of digital waveform data and to compare the validated mean value with protocol-specific and subject-specific normal data to assess for deviations between of the validated mean value of the digital waveform data and that of the protocol-specific and subject-specific normal data.

10. The DSSEP diagnostic apparatus of claim 1 wherein the software package is configured to drive the computer system to performed serially with respect to two or more different stimulation sites on the subject.

11. The DSSEP diagnostic apparatus of claim 1 wherein the software package is configured to drive the computer system to correlate a series of digital waveform data with electromyogram (EMG) data obtained from the same subject.

12. The DSSEP diagnostic apparatus of claim 1 wherein the software package is configured to drive the computer system to compare and to evaluate in real-time changes in the digital waveform data and to save the comparisons and changes as a function of time.

13. The DSSEP diagnostic apparatus of claim 1 wherein the software package is configured to drive the computer system to obtain a validated mean value from the digital waveform data set; and
to compare the validated mean value with a protocol-specific and subject-specific normal data so that deviations between the validated mean value from the normal data can be noted.

14. The DSSEP diagnostic apparatus of claim 1 wherein the software package is configured to drive the computer system to direct the switchbox to apply the electrical stimulus at another dermal stimulating site, to detect another evoked potential response at another dermal recording site, to record the detected another evoked potential response as another digital waveform data, and to average a series of the another digital waveform data and to compare a mean value of the series of the another digital waveform data against another protocol-specific and subject-specific normal data set.

15. The DSSEP diagnostic apparatus of claim 1 wherein the electrical stimulator is configured to be manually adjusted to adjust the electrical charge without exceeding a threshold to elicit a motor response.

16. The DSSEP diagnostic apparatus of claim 1 wherein the software package is configured to drive the computer system adjust the electrical charge from the electrical stimulator without exceeding a threshold to elicit a motor response.

17. The DSSEP diagnostic apparatus of claim 1 wherein the software package is configured to drive the computer system to select a protocol-specific and subject-specific normal data associated with a variety of different non-symptomatic population subject sources from the group consisting of height, limb length, limb temperature, species, race gender, age, and weight.

18. The DSSEP diagnostic apparatus of claim 1 wherein the software package is configured to drive the computer system to use a mathematical conditioning algorithm to obtain a validated mean value of a series of digital waveform data, and to assign a latency value and an amplitude value for the series of digital waveform data using a mathematical algorithm.

19. The DSSEP diagnosis apparatus of claim 1 wherein the software package is configured to drive the computer system to assess a difference between the mean validated data against normal data using a mathematical function selected from the group consisting of a first order mathematical function, a second order mathematical function and a third order mathematical function.

20. A kit for a dermatomal somatosensory evoked potential (DSSEP) diagnostic apparatus for evaluating nerve root functions in a mammalian subject, the kit comprising:
an electrical stimulator for generating an electrical stimulus;
a stimulus site selector switchbox configured to be coupled to the electrical stimulator;
a connection box configured to be coupled to the stimulus site selector switchbox;
a plurality of stimulating electrodes configured to be coupled to the connection box wherein the stimulating electrodes are configured to receive the electrical stimulus and configured to apply the electrical stimulus onto a dermal stimulation site of the mammalian subject;
a plurality of recording electrodes configured to be coupled to the connection box wherein the recording electrodes are configured to receive an evoked potential response at a dermal recording site of the mammalian subject such that the evoked potential response being induced by the applied electrical stimulus at the dermal stimulation site such that the evoked potential response passes through nerves from the dermal stimulation site to the dermal recording site;
a computer system configured to be coupled to the switchbox, to the connection box, to the stimulating electrodes and to the recording electrodes, the computer system comprising:
a digitizer for digitally converting the evoked potential response into a digital waveform data;
a memory for storing the digital waveform data; and
a display terminal configured to display the digital waveform data; and
a software package configured to drive the computer system to process the digital waveform data.
